(19) United States
(12) Patent Application Publication
(10) Pub. No.: US 2022/0096542 A1
(43) Pub. Date: Mar. 31, 2022

(54) AUGMENTATION OF NATURAL KILLER CELL ACTIVITY AND INDUCTION OF CYTOTOXIC IMMUNITY USING LEUKOCYTE LYSATE ACTIVATED ALLOGENEIC DENDRITIC CELLS: STEMVACS

(71) Applicant: THERAPEUTIC SOLUTIONS INTERNATIONAL, INC., Oceanside, CA (US)

(72) Inventors: Thomas E. ICHIM, Oceanside, CA (US); Timothy G. DIXON, Oceanside, CA (US); James VELTMEYER, Oceanside, CA (US)

(73) Assignee: THERAPEUTIC SOLUTIONS INTERNATIONAL, INC., Oceanside, CA (US)

(21) Appl. No.: 17/364,646

(22) Filed: Jun. 30, 2021

Related U.S. Application Data
(60) Provisional application No. 63/045,863, filed on Jun. 30, 2020.

(51) Int. Cl. A61K 35/15

(52) U.S. Cl. CPC A61K 35/15

(57) ABSTRACT

Stimulation of immunity would be beneficial in various chronic conditions such as viral infections and neoplasia. Autologous dendritic cell therapy has been widely described in the immunotherapy literature and has been approved by the FDA for treatment of prostate cancer. Unfortunately, the need to generate individual doses is costly and limited by ability of the patients to have sufficient starting cell numbers available to generate sufficient dendritic cells. Here we describe the process of preparing allogeneic dendritic cells utilizing a leukocyte lysate based approach. These data support development of StemVacs for conditions that would benefit from NK activation such as cancer and COVID-19.

### CROSS-REFERENCE TO RELATED APPLICATIONS

This application claims the benefit of priority to U.S. Provisional Application No. 63/045,863, filed Jun. 30, 2020, the contents of which are hereby incorporated by reference.

### FIELD OF THE INVENTION

The present invention pertains to the treatment of patients in need, such as those suffering from a viral infection like COVID-19 or a neoplasia, through the use leukocyte lysate activated allogenic dendritic cells.

### BACKGROUND

Acute Respiratory Distress Syndrome (ARDS) is a condition of acute respiratory failure caused by a variety of factors which are related to inflammation and release of various activators of the innate immune system such as cytokines and pyrogenic factors [1-9].

It is widely known that ARDS generally presents with progressive hypoxemia, dyspnea and increased work of breathing. Patients often require mechanical ventilation and supplemental oxygen [10]. Over the years, our understanding of ARDS has advanced significantly, with elucidation of several of the molecular and cellular pathways involved in initiation, progression and resolution/fibrosis. However, ARDS is still represents significant morbidity and mortality and therapeutic strategies to mitigate the foregoing have resulted in limited translational success. Part of this failure stems from the very different presentations of ARDS between people, as well as differences in their genetic composition.

ARDS is caused by many situations bacterial and viral pneumonia, sepsis, inhalation of harmful substances, head, chest or other major injury, burns, blood transfusions, near drowning, aspiration of gastric contents, pancreatitis, intravenous drug use, and abdominal trauma. Furthermore, those with a history of chronic alcoholism are at a higher risk of developing ARDS [11-13]. Alcoholism affects several parameters relevant to ARDS including: a) reduction in glutathione levels [14, 15], b) increasing levels of adhesion molecules on lung blood vessels so as to increase recruitment of inflammatory cells [16]; c) upregulating lung adenosine levels, resulting in impaired active Na(+) transport in the lung [17]; and d) suppression of pulmonary immunity [18].

One of the cardinal symptoms of ARDS is fluid accumulation in the lungs. When this occurs, the elastic air sacs (alveoli) in the lungs fill with fluid and the function of the alveoli is impaired. The result is that less oxygen reaches the bloodstream, depriving organs of the oxygen required for normal function and viability. In some instances, ARDS occurs in people who are already critically ill or who have significant injuries. Severe shortness of breath, the main symptom of ARDS, usually develops within a few hours to a few days after the precipitating injury or infection.

Unfortunately, many patients who develop ARDS do not survive. The risk of death increases with age and severity of illness. Of the people who do survive ARDS, some recover completely while others experience lasting damage to their lungs.

Currently there exist no effective pharmacologic therapies for treatment or prevention of ARDS. While inhibition of fibrin formation mitigated injury in some preclinical models of ARDS, anticoagulation therapies in humans do not attenuate ARDS and may even increase mortality. Protective lung ventilator strategies remain the mainstay of available treatment options. Due to the significant morbidity and mortality associated with ARDS and the lack of effective treatment options, new therapeutic agents for the treatment of ARDS and new treatment methods for ARDS are needed.

The present disclosure addresses the unmet need in the art by providing novel therapeutic agents useful in the treatment of ARDS which concurrently activate immunity, specifically NK mediated immunity and methods of treatment for ARDS and conditions related thereto through the administration of such novel therapeutic agents.

### SUMMARY

Preferred embodiments are directed to methods of concurrently stimulating natural killer cell activity while decreasing pulmonary inflammation through administration of a dendritic cell population activated by exposure to leukocyte lysate to a patient in need.

Preferred embodiments include methods wherein said dendritic cells are autologous.

Preferred embodiments include methods wherein said dendritic cells are allogeneic.

Preferred embodiments include methods wherein said dendritic cells are derived from bone marrow.

Preferred embodiments include methods wherein said dendritic cells are derived from umbilical cord.

Preferred embodiments include methods wherein said dendritic cells are derived from peripheral blood.

Preferred embodiments include methods wherein said dendritic cells are generated by treatment with IL-4 and GM-CSF.

Preferred embodiments include methods wherein said leukocyte lysate is obtained by freeze-thawing of leukocytes in a manner sufficient to cause rupture of the cellular membrane.

Preferred embodiments include methods wherein said leukocyte lysate is added to said dendritic cells at day 5 of culture.

Preferred embodiments include methods wherein said leukocyte lysate is added at a concentration of 1-10 micrograms of lysate protein to 1 million dendritic cells.

Preferred embodiments include methods wherein said leukocyte lysate is in contact with said dendritic cells for a period of 1 second to 14 days.

Preferred embodiments include methods wherein said leukocyte lysate is in contact with said dendritic cells for a period of 24 hours.

Preferred embodiments include methods wherein the patient in need is suffering from a neoplasia, and wherein said administration of the dendritic cell population is administered in an amount sufficient to treat said neoplasia.

Preferred embodiments include methods wherein said neoplasia is a melanoma.

Preferred embodiments include methods wherein the patient in need is suffering from a viral infection, and wherein said administration of the dendritic cell population is administered in an amount sufficient to treat said viral infection.

Preferred embodiments include methods wherein the viral infection is COVID-19.

### BRIEF DESCRIPTION OF THE DRAWINGS

FIG. 1 is a bar graph showing the effect of DCs activated by leukocyte extract on CD80 expression

FIG. 2 is a bar graph showing the effect of DCs activated by leukocyte extract on CD86 expression

FIG. 3 is a bar graph showing the effect of DCs activated by leukocyte extract on IL12 expression

FIG. 4 is a bar graph showing the effect of DCs activated by leukocyte extract on NK activity

FIG. 5 is a bar graph showing the effect of DCs activated by leukocyte extract in inhibiting B16 melanoma

FIG. 6 is a bar graph showing the effect of NK depletion on DCs activated by leukocyte extract in inhibiting B16 melanoma

FIG. 7 is a bar graph showing the effect of DCs activated by leukocyte extract in reducing lung inflammation.

### DETAILED DESCRIPTION OF THE INVENTION

In one embodiment of the invention, immunization to viruses of the same type the patient is suffering from is provided prior to cytotoxic, or immunogenic cell death induction of the virus. Immunization of the patient may be performed using known means in the art, using suitable adjuvants. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing said responses are well known in the art. In a preferred embodiment, antibody responses are assessed to a panel of virus associated proteins subsequent to immunization of patient. Antibody responses are utilized to guide which peptides will be utilized for prior immunization. For example, if a patient is immunized with viral antigen on a weekly basis, the subsequent assessment of antibody responses is performed at approximately 1-3 months after initiation of immunization. Protocols for immunization include weekly, biweekly, or monthly. Assessment of antibody responses is performed utilizing standard enzyme linked immunosorbent (ELISA) assay.

Host immunity to cancers has been extensively documented both in animal models and humans going back to the days of William Coley. In fact, there is strong evidence that the immune surveillance plays a critical role in limiting tumor outgrowth in the early stages of tumorigenesis, for example, this is way transplant recipients, who are under constant immune suppression have higher rates of cancer. However, the ability to prime tumor-specific T-cells and sustain an immune response that imparts a measurable clinical benefit, is limited in the setting of an established tumor burden. Taken together, these findings suggest numerous requirements for effective immunotherapy. Tumor-specific T cells must not only possess a sizeable precursor frequency and reach sufficient numbers following activation, but they must also be able to traffic to the tumor site and effectively kill their targets in situ.

One of the most potent cellular defense mechanisms against cancer is the natural killer (NK) cell. These cells, originally termed large granular lymphocytes (LGL) have the unique ability to spontaneously kill tumor cells without need for prior sensitization. It is believed that NK cells can selectively kill cancer tissue for two main reasons: a) cancer tissues express reduced levels of MHC I, and it is known that MHC I acts as a negative signal to NK cells to prevent their activation; and b) cancer cells express numerous “stress” proteins, these stress proteins stimulate NK activatory receptors such as NKG2D.

The utilization of NK cells therapeutically has been demonstrated since the 1980s with varying degrees of success. More recently, the NK cell line NK-92 has been used in numerous clinical trials. The importance of NK cells is that not only do their infiltration correlate with improved survival, but NK cells can synergize with other approaches such as vaccines and antibodies. In fact, numerous cancer antibodies dependent on an active NK cell compartment.

When NK cells are used for viral infections such as COVID-19, there is always a fear that the NK cells can be activating an excessive cytokine response, called “cytokine storm”, which causes acute respiratory distress syndrome, and even death. So on the one hand NK cells are important for antiviral immunity and the FDA has cleared a clinical trial using umbilical cord derived NK cells for treatment of COVID19, but on the other hand the fear of cytokine storm remains.

Dendritic cells are the more potent antigen presenting cell, being uniquely capable of inducing T cell responses in naïve cells. Additionally, numerous studies have shown efficacy of DC in stimulating anticancer immunity, with one DC therapy being approved by the FDA for treatment of prostate cancer.

We questioned whether dendritic cells (DC) can be used to stimulate NK cells. We demonstrated that replicating a “danger” environment by stimulating DC with leukocyte lysate is superior to conventional means of stimulating NK cells, and that this particular means of activating DC endows them ability to induce NK cell activation. This ability was demonstrated to confer immunity in a model of cancer in mouse.

Strikingly the leukocyte lysate activated dendritic cells produced less it-12 as compared to conventional dendritic cells and also reduced 1 ps induced lung inflammation.

### Example 1. Preparation of StemVacs

### IL-4 Reconstitution:

Reconstitute in PBS with 8% Human Albumin Solution to a final concentration of 20 μg/ml. (Example: We reconstituted 10 μg of IL-4 in 500 μL of solution)

### GM-CSF Reconstitution:

Reconstitute in PBS with 8% Human Albumin Solution to a final concentration of 20 μg/ml. (Example: We reconstituted 10 μg of GM-CSF in 500 μL of solution)

### Dendritic Cell Culture Media (Keep at 37 C for Culture):

RPMI 1640 w/L-glutamine and 25 mM HEPES1% Pen/Strep

5% Human Serum AB50 μM BME

Before culture make sure to filter with a 0.22 μm filter to remove bacterial contaminants. PBS (1×)—for washing platelets out of supernatant following Ficoll separation Procedure:

1. Obtain 10 ml of blood sample
2. Dilute 1:1 in PBS warmed to RT
3. Place remaining ˜180 ml PBS on ice

### 4. Underlay 10 ml Ficoll

a. Slow down pipette aid to minimize disruptance to interphase layer.
b. Be careful of air bubbles that could disrupt layer
   5. Carefully transfer tube into centrifuge being careful not to disrupt interphase layer
a. Spin at 2000 RPM, 20 min, no brakes @RT
b. After spin is completed set temperature on centrifuge to 4 C
   6. Remove interphase layer and transfer to new 50 ml conical
a. Optional tips: removing the upper layer (containing PRP) for easier access to interphase layer—this allows you to use a P1000 pipette instead of a pipette aid for greater accuracy
   7. Add PBS (on ice or 4 C) to solution containing interphase layer to bring the total volume to 50 ml
   8. Centrifuge at 1500 RPM, 10 min, brakes on @4 C
   9. Inspect clarity of supernatant, repeat wash cycles (900 RPM, 10 min, brakes, @4 C) until supernatant is clear (typically 3 wash cycles completed)
   10. Aspirate supernatant and resuspend in 1 ml DC Culture Media warmed to 37 C
   11. Take 10 μL of cell suspension and count using a hemocytometer to determine the number of wells necessary for culture (approximately 8 million mononuclear cells per well)
a. Add trypan blue to check cell viability
   12. Aliquote cells to a 6 well plate bringing the total volume per well to ˜3 ml
   13. Place 6 well plate in incubator at 37 C, 5% CO2 for 1 hour
   14. After hour incubation is complete, aspirate supernatant leaving behind adherent cells
   15. Replace with fresh DC Culture Media with 3 ml per well
   16. IL-4 concentration
a. Desired final concentration 20 ng/ml
b. Therefore given a stock solution of 20 μg/ml, add 3 μL per well
   17. GM-CSF concentration
a. Desired final concentration 100 ng/ml
b. Therefore given a stock solution of 20 μg/ml, add 15 μL per well
   18. Place 6 well plate back in incubator at 37 C with 5% CO2
   19. On day 2 after isolation procedure, replace cytokines
   20. On day 4 after isolation procedure, replace cytokines
   21. On 5^{th} day, add maturation signal for 1 day
a. 100 ng/ml Lipopolysaccharides
   22. On 6^{th} day, add Leukocyte lysate 1 μL
   23. After 24 h collect cells in a 50 ml tube and wash at 900 rpm, 10 min, brake, @4 C.
   24. Aspirate supernatant and resuspend cells in 3 ml PBS 1× or saline solution 0.9%. Add to IV infusion solution.

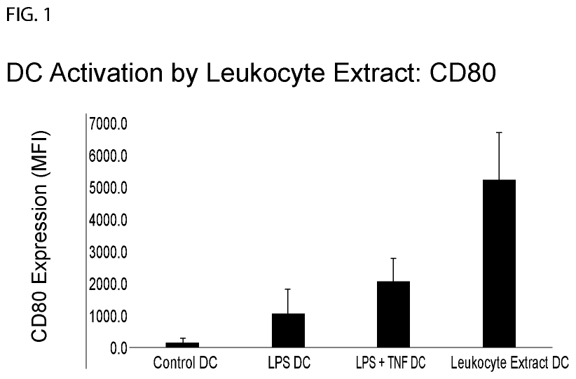

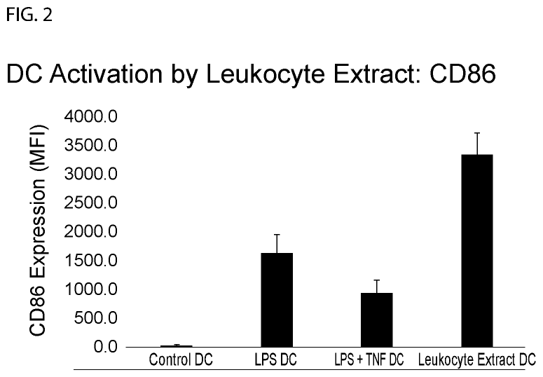

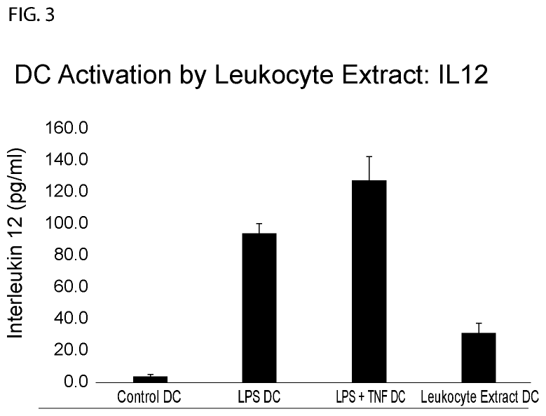

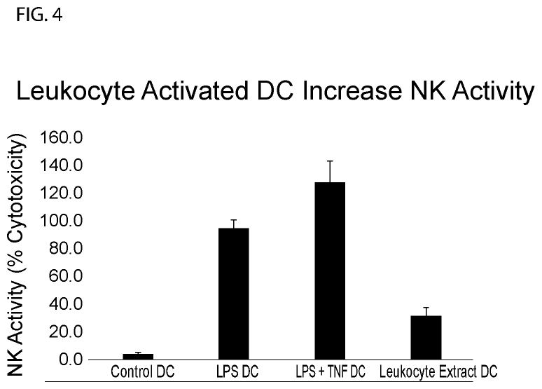

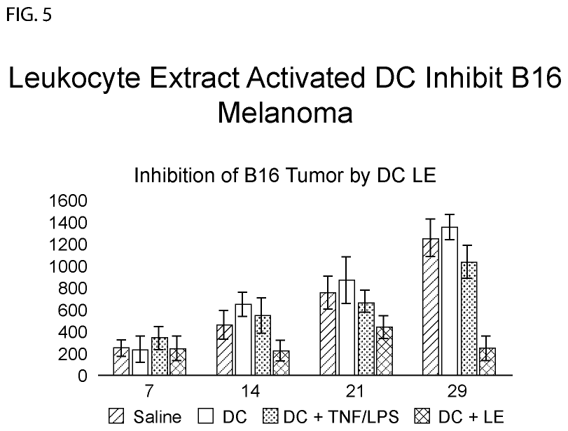

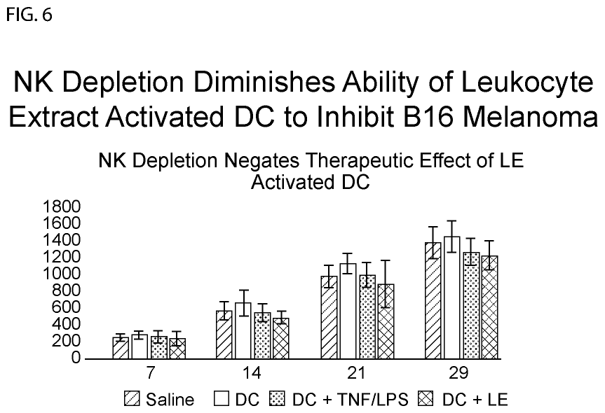

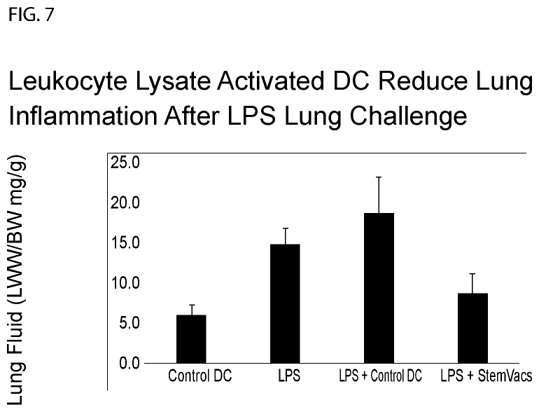

Results are shown in FIGS. 1-7.

### REFERENCES

1. Balamayooran T, Balamayooran G, Jeyaseelan S: Review: Toll-like receptors and NOD-likereceptors in pulmonary antibacterial immunity Innate Immun 2010, 16(3):201-210.
2. Oppeltz R F, Rani M, Zhang Q, Schwacha M G: Burn-induced alterations in toll-like receptor-mediated responses by bronchoalveolar lavage cells. Cytokine 2011, 55(3):396-401.
3. Dolinay T, Kim Y S, Howrylak J, Hunninghake G M, An C H, Fredenburgh L, Massaro A F, Rogers A, Gazourian L, Nakahira K et al: Inflammasome-regulated cytokines are critical mediators of acute lung injury. Am J Respir Crit Care Med 2012, 185(11):1225-1234.
4. de Pablo R, Monserrat J, Prieto A, Alvarez-Mon M: Role of circulating soluble chemokines inseptic shock. Med Intensiva 2013, 37(8):510-518.
5. Parikh S M: Dysregulation of the angiopoietin-Tie-2 axis in sepsis and ARDS. Virulence 2013, 4(6):517-524.
6. Nakahira K, Kyung S Y, Rogers A J, Gazourian L, Youn S, Massaro A F, Quintana C, Osorio J C, Wang Z, Zhao Y et al: Circulating mitochondrial DNA in patients in the ICU as a marker of mortality: derivation and validation. PLoS Med 2013, 10(12):e1001577; discussion e1001577.
7. Tang L, Bai J, Chung C S, Lomas-Neira J, Chen Y, Huang X, Ayala A: Active players in resolution of shock/sepsis induced indirect lung injury: immunomodulatory effects of Tregsand PD-1. J Leukoc Biol 2014, 96(5):809-820.
8. Lefrancais E, Mallavia B, Zhuo H, Calfee C S, Looney M R: Maladaptive role of neutrophilextracellular traps in pathogen-induced lung injury. JCI Insight 2018, 3(3).
9. Yuan Z, Bedi B, Sadikot R T: Bronchoalveolar Lavage Exosomes in Lipopolysaccharide-induced Septic Lung Injury. J Vis Exp 2018(135).
10. Thompson B T, Chambers R C, Liu K D: Acute Respiratory Distress Syndrome. N Engl J Med, 2017, 377(6):562-572.
11. Moss M, Bucher B, Moore F A, Moore E E, Parsons P E: The role of chronic alcohol abuse in the development of acute respiratory distress syndrome in adults. JAMA 1996, 275(1):50-54.
12. Moss M, Steinberg K P, Guidot D M, Duhon G F, Treece P, Wolken R, Hudson L D, Parsons P E: The effect of chronic alcohol abuse on the incidence of ARDS and the severity of the multiple organ dysfunction syndrome in adults with septic shock: an interim and multivariate analysis. Chest 1999, 116(1 Suppl):97S-98S.
13. Guidot D M, Hart C M: Alcohol abuse and acute lung injury: epidemiology and pathophysiology of a recently recognized association. J Investig Med 2005, 53(5):235-245.
14. Moss M, Guidot D M, Wong-Lambertina M, Ten Hoor T, Perez R L, Brown L A: The effects of chronic alcohol abuse on pulmonary glutathione homeostasis. Am J Respir Crit Care Med 2000, 161(2 Pt 1):414-419.
15. Liang Y, Yeligar S M, Brown L A: Chronic-alcohol-abuse-induced oxidative stress in the development of acute respiratory distress syndrome. Scientific World Journal 2012, 2012:740308.
16. Burnham E L, Moss M, Harris F, Brown L A: Elevated plasma and lung endothelial selectin levels in patients with acute respiratory distress syndrome and a history of chronic alcohol abuse. Crit Care Med 2004, 32(3):675-679.
17. Dada L, Gonzalez A R, Urich D, Soberanes S, Manghi T S, Chiarella S E, Chandel N S, Budinger G R, Mutlu G M: Alcohol worsens acute lung injury by inhibiting alveolar sodium transport through the adenosine A1 receptor. PLoS One 2012, 7(1):e30448.
18. Kaphalia L, Calhoun W J: Alcoholic lung injury: metabolic, biochemical and immunological aspects. Toxicol Lett 2013, 222(2):171-179.

1. A method of concurrently stimulating natural killer cell activity while decreasing pulmonary inflammation through administration of a dendritic cell population activated by exposure to leukocyte lysate to a patient in need.

2. The method of claim 1, wherein said dendritic cells are autologous.

3. The method of claim 1, wherein said dendritic cells are allogeneic.

4. The method of claim 1, wherein said dendritic cells are derived from bone marrow.

5. The method of claim 1, wherein said dendritic cells are derived from umbilical cord.

6. The method of claim 1, wherein said dendritic cells are derived from peripheral blood.

7. The method of claim 1, wherein said dendritic cells are generated by treatment with IL-4 and GM-CSF.

8. The method of claim 1, wherein said leukocyte lysate is obtained by freeze-thawing of leukocytes in a manner sufficient to cause rupture of the cellular membrane.

9. The method of claim 1, wherein said leukocyte lysate is added to said dendritic cells at day 5 of culture.

10. The method of claim 9, wherein said leukocyte lysate is added at a concentration of 1-10 micrograms of lysate protein to 1 million dendritic cells.

11. The method of claim 10, wherein said leukocyte lysate is in contact with said dendritic cells for a period of 1 second to 14 days.

12. The method of claim 10, wherein said leukocyte lysate is in contact with said dendritic cells for a period of 24 hours.

13. The method of claim 1, wherein the patient in need is suffering from a neoplasia, and wherein said administration of the dendritic cell population is administered in an amount sufficient to treat said neoplasia.

14. The method of claim 13, wherein said neoplasia is a melanoma.

15. The method of claim 1, wherein the patient in need is suffering from a viral infection, and wherein said administration of the dendritic cell population is administered in an amount sufficient to treat said viral infection.

16. The method of claim 15, wherein the viral infection is COVID-19.
